(19) United States
(12) Patent Application Publication
(10) Pub. No.: US 2003/0143664 A1
(43) Pub. Date: Jul. 31, 2003

(54) Products of manufacture and processes for peptide synthesis

(75) Inventors: Grace Desantis, San Diego, CA (US); Mark J. Burk, San Diego, CA (US)

Correspondence Address: GREGORY P. EINHORN, Fish & Richardson P.C., Suite 500, 4350 La Jolla Village Drive, San Diego, CA, 92122, US

(73) Assignee: Diversa Corporation, San Diego, CA (US)

(21) Appl. No.: 10/309,758

(22) Filed: Dec. 3, 2002

Related U.S. Application Data
(60) Provisional application No. 60/336,580, filed on Dec. 3, 2001.

(51) Int. Cl. C12P 21/06

(52) U.S. Cl. 435/68.1

(57) ABSTRACT

In one aspect, the invention provides a nontemplate directed, enzymatic thermo-cycled (NTDET) peptide synthesis process. The invention also provides products of manufacture comprising a reaction chamber for synthesizing a peptide or a polypeptide.

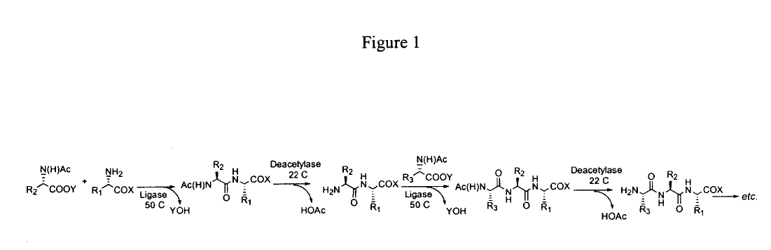

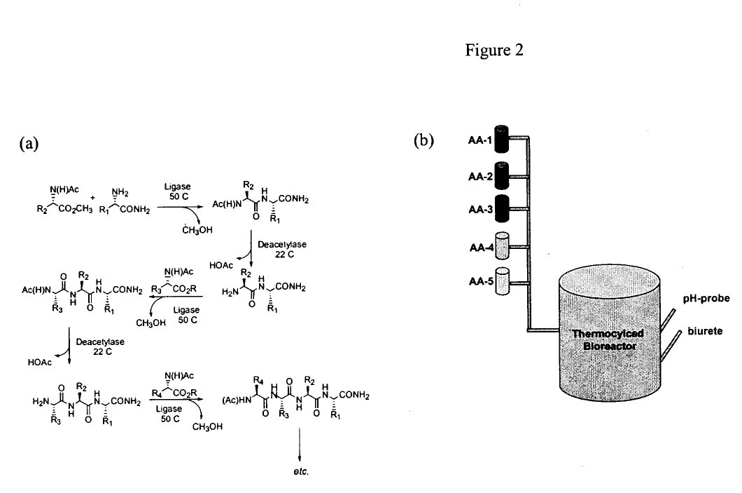

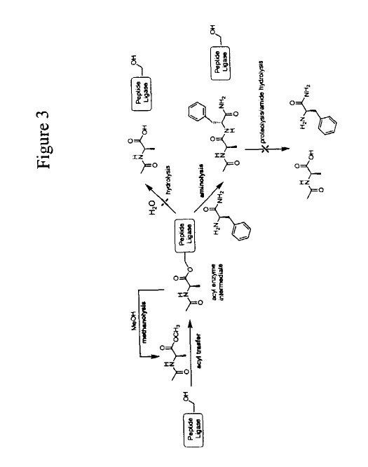

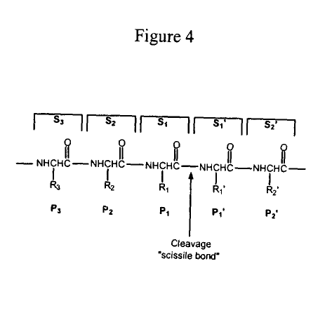

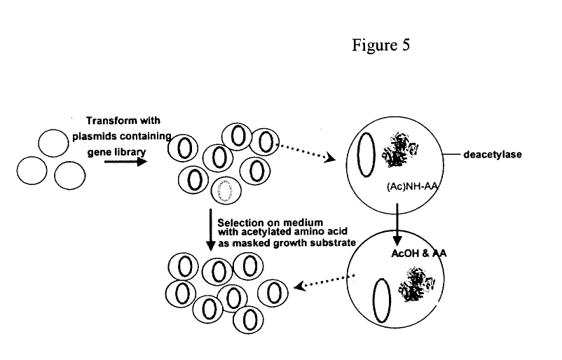

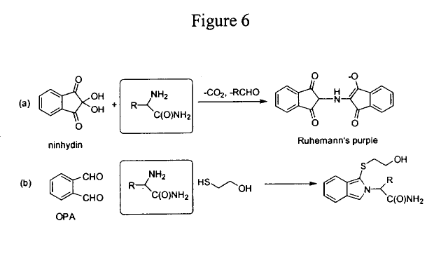

### CROSS-REFERENCES TO RELATED APPLICATIONS

The present application claims priority to U.S. Provisional Application 60/336,580, filed Dec. 3, 2001. The aforementioned application is explicitly incorporated herein by reference in its entirety and for all purposes.

### TECHNICAL FIELD

This invention generally relates to synthetic and pharmaceutical chemistry. In one aspect, the invention provides a nontemplate directed, enzymatic thermo-cycled (NTDET) peptide synthesis process.

### BACKGROUND

Peptides exhibit biological functions as diverse as sexual maturation and reproduction, blood pressure regulation, glucose metabolism, thermal control, enzyme inhibition and analgesia. Accordingly, peptides are a viable treatment for many diseases. Currently marketed peptide drugs address important therapeutic areas such prostate cancer and multiple sclerosis. However, the exorbitant costs of peptide drugs substantially limits patient access to them. One of the principal reasons for their high cost is due to the challenges of peptide synthesis. The preparation of very small peptides, up to 4 amino acids, is synthetically tractable. The preparation of peptides composed of greater than about 30 natural amino acids may be achieved via recombinant expression techniques. However, peptides of 5 to 30 amino acids are very difficult to prepare by current methods on large scale.

### SUMMARY

The invention provides enzymatic processes for synthesizing a peptide comprising the following steps: (a) providing at least two amino acids, or, providing at least one peptide and at least one amino acid, or, providing at least two peptides; (b) providing a peptide ligase and a deacetylase, wherein the peptide ligase and the deacetylase are active under different reaction conditions and the peptide ligase is active after exposure to reaction conditions where the deacetylase is active and the deacetylase is active after exposure to reaction conditions where the peptide ligase is active; (c) contacting the amino acids of step (a), or the peptide and the amino acid of step (a), or the peptides of step (a) with the peptide ligase of step (b) under conditions wherein the peptide ligase catalyzes the formation of a peptide bond between the amino acids or between the peptide and the amino acid or between the peptides, thus making at least a dipeptide; and (d) contacting the peptide of step (c) with the deacetylase of step (b) under conditions wherein the deacetylase catalyzes the deacylation of the peptide, thereby synthesizing a peptide or a polypeptide.

The invention provides enzymatic processes for synthesizing a peptide comprising the following steps: (a) providing reaction chamber comprising a peptide ligase and a deacetylase, wherein the peptide ligase and the deacetylase are active under different reaction conditions and the peptide ligase is active after exposure to reaction conditions where the deacetylase is active and the deacetylase is active after exposure to reaction conditions where the peptide ligase is active; (b) adding at least two amino acids, or at least an amino acid and a peptide, or at least two peptides to the reaction chamber under conditions wherein the peptide ligase is active and the peptide ligase catalyzes the formation of a peptide bond between the amino acids, or between the peptide and the amino acid, or between the peptides; and, (c) changing the conditions in the reaction chamber to conditions wherein the deacetylase is active and the deacetylase catalyzes the deacylation of the peptide formed in step (b).

The enzymatic processes can further comprise changing conditions in the reaction chamber to conditions wherein the peptide ligase is active and the deacetylase is inactive and adding at least one additional amino acid or peptide to the reaction chamber. The enzymatic processes can further comprise changing conditions in the reaction chamber to conditions such that the deacetylase is active. In alternative aspects, the temperature conditions, the pH conditions, the salt conditions, the buffer conditions, the humidity conditions are changed in the reaction chamber. In one aspect, the peptide ligase and the deacetylase are active at different conditions, e.g., temperature conditions, pH conditions, salt conditions, buffer conditions, humidity conditions and the like.

The enzymatic process can further comprise reiterating the process, thereby making a longer peptide or polypeptide. The reiterated process can comprise cycling the reaction conditions, e.g., temperature conditions, pH conditions, salt conditions, buffer conditions, humidity conditions and the like. In one aspect, the reiterated process can comprise thermocycling the peptide ligase and the deacylation reactions. In one aspect, the reaction chamber comprises a thermocycled bioreactor. In one aspect, the peptide ligase and the deacetylase are active at different pH conditions. In one aspect, the peptide ligase and the deacetylase are active at different salt conditions. In one aspect, the peptide ligase and the deacetylase are active at different solute conditions.

In alternative aspects, the reaction chamber comprises a tube, a well, a capillary, e.g., a capillary array, such as a GIGAMATRIX™ capillary array. In alternative aspects, the peptide ligase, the deacetylase or both are immobilized, e.g., in the reaction chamber.

In alternative aspects, the peptide or polypeptide is about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70 or more peptides in length, or between about 2 and about 50 peptides in length, 3 and about 30 peptides in length or 5 and about 25 peptides in length.

In one aspect, the peptide ligase is active at a higher temperature than the deacetylase. In one aspect, the deacetylase is inactive and thermotolerant in the conditions set for the peptide ligase activity. In one aspect, the ligase is inactive and thermotolerant in the conditions set for the deacetylase activity. In one aspect, the amino acids, or the peptide and the amino acid, are contacted with the peptide ligase under conditions comprising about a temperature of about 45° C., 50° C., 55° C., 60° C., 65° C., 70° C. or 75° C. or higher. In one aspect, the deacetylase reaction conditions comprise a temperature of about 20° C. or lower, 25° C., 30° C., 35° C. or 40° C. In one aspect, the peptide ligase and the deacetylase reactions are thermocycled, e.g., between about 50° C. and about 20° C.

In one aspect, at least one peptide is a naturally occurring L-amino acid. In one aspect, at least one peptide is a glycosylated amino acid. In one aspect, at least one peptide is a phosphorylated amino acid. In one aspect, at least one peptide is a non-naturally occurring amino acid. In one aspect, the peptide is a D-amino acid. In one aspect, at least one peptide is a non-naturally occurring aromatic amino acid. In one aspect, the non-naturally occurring aromatic amino acid comprises a D- or L- naphylalanine, a D- or L-phenylglycine, a D- or L-2 thieneylalanine, a D- or L-1, -2, 3-, or 4- pyreneylalanine, a D- or L-3 thieneylalanine, a D- or L-(2-pyridinyl)-alanine, a D- or L-(3-pyridinyl)-alanine, a D- or L-(2-pyrazinyl)-alanine, a D- or L-(4-isopropyl)-phenylglycine, a D-(trifluoromethyl)-phenylglycine, a D-(trifluoromethyl)-phenylalanine, a D-p-fluoro-phenylalanine, a D- or L-p-biphenylphenylalanine, a D- or L-p-methoxy-biphenylphenylalanine; D- or L-2-indole(alkyl)alanines. In one aspect, the non-naturally occurring aromatic amino acid comprises a thiazolyl, a thiophenyl, a pyrazolyl, a benzimidazolyl, a naphthyl, a furanyl, a pyrrolyl or a pyridyl aromatic ring. In one aspect, the non-naturally occurring amino acid comprises a D- or L-alkylainine. In one aspect, the alkyl of the alkylainines comprises a substituted or unsubstituted methyl, ethyl, propyl, hexyl, butyl, pentyl, isopropyl, iso-butyl, sec-isotyl or iso-pentyl.

In one aspect, the peptide ligase is a hydrolase, such as a serine hydrolase. In one aspect, the peptide ligase is an esterase, a peptide synthetase, such as a muramoyl peptide synthetase or a lipase. In one aspect, the process can further comprise use of at least two peptide ligases. In one aspect, the peptide ligase is a catalytic antibody.

In one aspect, the deacetylase is an aminoacylase. In one aspect, the deacetylase is a D-aminoacylase or an L-aminoacylase. In one aspect, the process further comprises use of at least two aminoacylases. In one aspect, the deacetylase is a catalytic antibody.

In one aspect, the reaction is driven in favor of peptide catalysis and reducing aminolysis by reacting the peptide ligase under conditions comprising a low water environment. In one aspect, the reaction is driven in favor of peptide catalysis and reducing aminolysis by reacting the peptide ligase under conditions comprising an organic solvent. In one aspect, the reaction is driven in favor of peptide catalysis and reducing aminolysis by reacting the peptide ligase under conditions comprising a substantially pure organic solvent. In one aspect, the reaction is driven in favor of peptide catalysis and reducing aminolysis by reacting the peptide ligase in a water and ethanol solvent. In one aspect, the reaction is driven in favor of peptide catalysis and reducing aminolysis by reacting the peptide ligase in pure methanol solvent or a pure ethanol solvent. In one aspect, the reaction is driven in favor of peptide catalysis and reducing aminolysis by reacting the peptide ligase under conditions comprising removing the product upon its formation by any means, e.g., by precipitation or by liquid-liquid extraction.

In one aspect, the process further comprises the injection of fresh enzyme into the reaction. The process can further comprise the injection of fresh enzyme into the reaction after each reiterated cycle. The process can further comprise the injection of fresh peptide ligase into the reaction. The process can further comprise the injection of fresh deacetylase into the reaction.

The invention provides enzymatic processes for synthesizing a peptide comprising the following steps (a) providing reaction chamber comprising a peptide ligase and a deacetylase, wherein the peptide ligase and the deacetylase are active at different temperatures and the peptide ligase is active after exposure to the deacetylase's activity temperature and the deacetylase is active after exposure to the peptide ligase's activity temperature, and at least two amino acids, or at least an amino acid and a peptide, or at least two peptides; (b) reacting the reaction chamber under conditions wherein the peptide ligase is active and the deacetylase is inactive and the peptide ligase catalyzes the formation of a peptide bond between the amino acids, or between the peptide and the amino acid, or between the peptides; and (c) changing the conditions in the reaction chamber to conditions wherein the deacetylase is active and the peptide ligase is inactive and the deacetylase catalyzes the deacylation of the peptide formed in step (b).

The invention provides products of manufacture comprising a reaction chamber for synthesizing a peptide comprising a peptide ligase and a deacetylase, wherein the peptide ligase and the deacetylase are active under different reaction conditions and the peptide ligase is active after exposure to reaction conditions where the deacetylase is active and the deacetylase is active after exposure to reaction conditions where the peptide ligase is active. In one aspect, the peptide ligase is immobilized. In one aspect, the deacetylase is immobilized.

In one aspect, the reaction chamber comprises a thermocycler. In one aspect, the reaction chamber comprises a capillary array such as a GIGAMATRIX™.

In one aspect, the reaction chamber is operably linked to an HPLC, a mass spectograph (MS), a liquid chromatograph (LC), and/or a multiplex interfaced liquid chromatograph (LC)-mass spectograph (MS) (LC-MS) system.

In one aspect, the reaction chamber further comprises a desorption/ionization device. In one aspect, the process further comprises an input for injection of enzyme or starting material into the reaction chamber.

What is claimed is:

1. An enzymatic process for synthesizing a peptide comprising the following steps
(a) providing at least two amino acids, at least one peptide and at least one amino acid, or at least two peptides;
(b) providing a peptide ligase and a deacetylase, wherein the peptide ligase and the deacetylase are active under different reaction conditions and the peptide ligase is active after exposure to reaction conditions where the deacetylase is active and the deacetylase is active after exposure to reaction conditions where the peptide ligase is active;
(c) contacting the amino acids of step (a), or the peptide and the amino acid of step (a), or the peptides of step (a), with the peptide ligase of step (b) under conditions wherein the peptide ligase catalyzes the formation of a peptide bond between the amino acids or between the peptide and the amino acid, or between the peptides, thus making at least a dipeptide; and
(d) contacting the peptide of step (c) with the deacetylase of step (b) under conditions wherein the deacetylase catalyzes the deacylation of the peptide, thereby synthesizing a peptide or a polypeptide.

2. An enzymatic process for synthesizing a peptide comprising the following steps
(a) providing reaction chamber comprising a peptide ligase and a deacetylase, wherein the peptide ligase and the deacetylase are active under different reaction conditions and the peptide ligase is active after exposure to reaction conditions where the deacetylase is active and the deacetylase is active after exposure to reaction conditions where the peptide ligase is active;
(b) adding at least two amino acids, at least an amino acid and a peptide, or at least two peptides to the reaction chamber under conditions wherein the peptide ligase is active and the peptide ligase catalyzes the formation of a peptide bond between the amino acids, or between the peptide and the amino acid, or between the peptides; and
(c) changing the conditions in the reaction chamber to conditions wherein the deacetylase is active and the deacetylase catalyzes the deacylation of the peptide formed in step (b).

3. The enzymatic process of claim 2, further comprising changing conditions in the reaction chamber to conditions wherein the peptide ligase is active and the deacetylase is inactive and adding at least one additional amino acid or peptide to the reaction chamber.

4. The enzymatic process of claim 3, further comprising changing conditions in the reaction chamber to conditions such that the deacetylase is active.

5. The enzymatic process of claim 4, wherein the temperature conditions are changed in the reaction chamber.

6. The enzymatic process of claim 4, further comprising reiterating the process, thereby making a longer peptide or polypeptide.

7. The enzymatic process of claim 1 or claim 2, wherein the peptide ligase and the deacetylase are active at different temperature conditions.

8. The enzymatic process of claim 5, wherein the reiterated process comprises thermocycling the peptide ligase and the deacylation reactions.

9. The enzymatic process of claim 5, wherein the reaction chamber comprises a thermocycled bioreactor.

10. The enzymatic process of claim 1 or claim 2, wherein the peptide ligase and the deacetylase are active at different pH conditions.

11. The enzymatic process of claim 1 or claim 2, wherein the peptide ligase and the deacetylase are active at different salt conditions.

12. The enzymatic process of claim 1 or claim 2, wherein the peptide ligase and the deacetylase are active at different solute conditions.

13. The enzymatic process of claim 2, wherein the reaction chamber comprises a capillary array.

14. The enzymatic process of claim 13, wherein the capillary array is GIGAMATRIX™.

15. The enzymatic process of claim 1 or claim 2, wherein the peptide ligase and the deacetylase are immobilized.

16. The enzymatic process of claim 1 or claim 2, wherein the peptide or polypeptide is between 2 and about 50 peptides in length.

17. The enzymatic process of claim 16, wherein the peptide or polypeptide is between 3 and about 30 peptides in length.

18. The enzymatic process of claim 17, wherein the peptide or polypeptide is between about 5 and about 25 peptides in length.

19. The enzymatic process of claim 1 or claim 2, wherein the peptide ligase is active at a higher temperature than the deacetylase.

20. The enzymatic process of claim 19, wherein the deacetylase is inactive and thermotolerant in the conditions set for the peptide ligase activity.

21. The enzymatic process of claim 1 or claim 2, wherein the amino acids, or the peptide and the amino acid, are contacted with the peptide ligase under conditions comprising about a temperature of about 50° C.

22. The enzymatic process of claim 1 or claim 2, wherein the deacetylase reaction conditions comprise a temperature of about 20° C.

23. The enzymatic process of claim 8, wherein the peptide ligase and the deacetylase reactions are thermocycled between about 50° C. and about 20° C.

24. The enzymatic process of claim 1 or claim 2, wherein at least one peptide is a naturally occurring L-amino acid.

25. The enzymatic process of claim 1 or claim 2, wherein at least one peptide is a glycosylated amino acid.

26. The enzymatic process of claim 1 or claim 2, wherein at least one peptide is a phosphorylated amino acid.

27. The enzymatic process of claim 1 or claim 2, wherein at least one peptide is a non-naturally occurring amino acid.

28. The enzymatic process of claim 27, wherein the peptide is a D-amino acid.

29. The enzymatic process of claim 27, wherein at least one peptide is a non-naturally occurring aromatic amino acid.

30. The enzymatic process of claim 29, wherein the non-naturally occurring aromatic amino acid comprises a D- or L- naphylalanine, a D- or L- phenylglycine, a D- or L-2 thieneylalanine, a D- or L-1, -2, 3-, or 4- pyreneylalanine, a D- or L-3 thieneylalanine, a D- or L-(2-pyridinyl)-alanine, a D- or L-(3-pyridinyl)-alanine, a D- or L-(2-pyrazinyl)-alanine, a D- or L-(4-isopropyl)-phenylglycine, a D-(trifluoromethyl)-phenylglycine, a D-(trifluoromethyl)-phenylalanine, a D-p-fluoro-phenylalanine, a D- or L-p-biphenylphenylalanine, a D- or L-p-methoxy-biphenylphenylalanine; D- or L-2-indole(alkyl)alanines.

31. The enzymatic process of claim 29, wherein the non-naturally occurring aromatic amino acid comprises a thiazolyl, a thiophenyl, a pyrazolyl, a benzimidazolyl, a naphthyl, a furanyl, a pyrrolyl or a pyridyl aromatic ring.

32. The enzymatic process of claim 27, wherein the non-naturally occurring amino acid comprises a D- or L-alkylainine.

33. The enzymatic process of claim 32, wherein the alkyl of the alkylainines comprises a substituted or unsubstituted methyl, ethyl, propyl, hexyl, butyl, pentyl, isopropyl, iso-butyl, sec-isotyl or iso-pentyl.

34. The enzymatic process of claim 1 or claim 2, wherein the peptide ligase is a hydrolase.

35. The enzymatic process of claim 34, wherein the hydrolase is a serine hydrolase.

36. The enzymatic process of claim 1 or claim 2, wherein the peptide ligase is an esterase or a lipase.

37. The enzymatic process of claim 1 or claim 2, wherein the peptide ligase is a muramoyl peptide synthetase.

38. The enzymatic process of claim 1 or claim 2, further comprising use of at least two peptide ligases.

39. The enzymatic process of claim 1 or claim 2, wherein the peptide ligase is a catalytic antibody.

40. The enzymatic process of claim 1 or claim 2, wherein the deacetylase is an aminoacylase.

41. The enzymatic process of claim 40, wherein the deacetylase is a D-aminoacylase or an L-aminoacylase.

42. The enzymatic process of claim 1 or claim 2, further comprising use of at least two aminoacylases.

43. The enzymatic process of claim 1 or claim 2, wherein the deacetylase is a catalytic antibody.

44. The enzymatic process of claim 1 or claim 2, wherein the reaction is driven in favor of peptide catalysis and reducing aminolysis by reacting the peptide ligase under conditions comprising a low water environment.

45. The enzymatic process of claim 1 or claim 2, wherein the reaction is driven in favor of peptide catalysis and reducing aminolysis by reacting the peptide ligase under conditions comprising an organic solvent.

46. The enzymatic process of claim 45, wherein the reaction is driven in favor of peptide catalysis and reducing aminolysis by reacting the peptide ligase under conditions comprising a substantially pure organic solvent.

47. The enzymatic process of claim 1 or claim 2, wherein the reaction is driven in favor of peptide catalysis and reducing aminolysis by reacting the peptide ligase in a water and ethanol solvent.

48. The enzymatic process of claim 1 or claim 2, wherein the reaction is driven in favor of peptide catalysis and reducing aminolysis by reacting the peptide ligase in pure methanol solvent or a pure ethanol solvent.

49. The enzymatic process of claim 1 or claim 2, wherein the reaction is driven in favor of peptide catalysis and reducing aminolysis by reacting the peptide ligase under conditions comprising removing the product upon its formation.

50. The enzymatic process of claim 49, wherein the reaction is driven in favor of peptide catalysis and reducing aminolysis by reacting the peptide ligase under conditions comprising removing the product upon its formation by precipitation or by liquid-liquid extraction.

51. The enzymatic process of claim 6, further comprising the injection of fresh enzyme into the reaction.

52. The enzymatic process of claim 51, further comprising the injection of fresh enzyme into the reaction after each reiterated cycle.

53. The enzymatic process of claim 51, further comprising the injection of fresh peptide ligase into the reaction.

54. The enzymatic process of claim 51, further comprising the injection of fresh deacetylase into the reaction.

55. An enzymatic process for synthesizing a peptide comprising the following steps
(a) providing reaction chamber comprising a peptide ligase and a deacetylase, wherein the peptide ligase and the deacetylase are active at different temperatures and the peptide ligase is active after exposure to the deacetylase's activity temperature and the deacetylase is active after exposure to the peptide ligase's activity temperature, and at least two amino acids, or at least an amino acid and a peptide, or at least two peptides,
(b) reacting the reaction chamber under conditions wherein the peptide ligase is active and the deacetylase is inactive and the peptide ligase catalyzes the formation of a peptide bond between the amino acids, or between the peptide and the amino acid, or between the peptides; and
(c) changing the conditions in the reaction chamber to conditions wherein the deacetylase is active and the peptide ligase is inactive and the deacetylase catalyzes the deacylation of the peptide formed in step (b).

56. A product of manufacture comprising a reaction chamber for synthesizing a peptide comprising a peptide ligase and a deacetylase, wherein the peptide ligase and the deacetylase are active under different reaction conditions and the peptide ligase is active after exposure to reaction conditions where the deacetylase is active and the deacetylase is active after exposure to reaction conditions where the peptide ligase is active.

57. The product of manufacture of claim 56, wherein the peptide ligase is immobilized.

58. The product of manufacture of claim 56, wherein the deacetylase is immobilized.

59. The product of manufacture of claim 56, wherein the reaction chamber comprises a thermocycler.

60. The product of manufacture of claim 56, wherein the reaction chamber comprises a capillary array.

61. The product of manufacture of claim 60, wherein the capillary array is GIGAMATRIX™.

62. The product of manufacture of claim 56, wherein the reaction chamber is operably linked to an HPLC.

63. The product of manufacture of claim 56, wherein the reaction chamber is operably linked to a mass spectograph (MS).

64. The product of manufacture of claim 56, wherein the reaction chamber is operably linked to a liquid chromatograph (LC).

65. The product of manufacture of claim 56, wherein the reaction chamber is operably linked to a multiplex interfaced liquid chromatograph (LC)-mass spectograph (MS) (LC-MS) system.

66. The product of manufacture of claim 56, wherein the reaction chamber further comprises a desorption/ionization device.

67. The product of manufacture of claim 56, further comprising an input for injection of enzyme or starting material into the reaction chamber.

68. A product of manufacture for high throughput robotic assays comprising a reaction chamber for synthesizing a peptide comprising an immobilized peptide ligase and an immobilized deacetylase, wherein the peptide ligase and the deacetylase are active under different reaction conditions and the peptide ligase is active after exposure to reaction conditions where the deacetylase is active and the deacetylase is active after exposure to reaction conditions where the peptide ligase is active.

69. The product of manufacture of claim 68, further comprising robotic arms to move microtiter plates between different platform components.

70. The product of manufacture of claim 68, further comprising temperature and humidity controlled incubators, liquid handling devices, bar-coding devices or plate readers.

71. The product of manufacture of claim 68, wherein the peptide ligase is an esterase or a lipase.

72. The product of manufacture of claim 68, wherein the peptide ligase is a muramoyl peptide synthetase.

73. The product of manufacture of claim 68, further comprising use of at least two peptide ligases.
